(19) United States
(12) Patent Application Publication
(10) Pub. No.: US 2016/0220121 A1
(43) Pub. Date: Aug. 4, 2016

(54) METHOD AND APPARATUS FOR THE MEASUREMENT OF TEMPERATURE DURING TREATMENT USING NEURAL SENSING

(71) Applicant: LUMENIS LTD., Yokneam Ilit (IL)

(72) Inventor: Israel Schuster, Kiryat-Tivon (IL)

(21) Appl. No.: 15/010,133

(22) Filed: Jan. 29, 2016

Related U.S. Application Data
(60) Provisional application No. 62/110,552, filed on Feb. 1, 2015.

(51) Int. Cl. A61B 5/01; A61B 5/00; A61N 5/06; A61B 5/04

(52) U.S. Cl. CPC A61B 5/01; A61B 5/04001; A61B 5/7278; A61N 5/0616; A61N 2005/0626

(57) ABSTRACT

In an aspect, a method for dynamically measuring temperature variations in skin tissue includes providing one or more electrical signal pickup elements on the skin tissue; providing at least one thresholding device to generate one or more agitating signals; providing at least one agitating device on the skin tissue at least some distance from the one or more electrical pickup signal elements; the method comprising: stimulating the skin tissue by applying one or more agitating signals to the agitating element until a neural response is indicated on a display, the neurons responding with a specific frequency modulation; identifying the relevant neural signal by locking on the modulation period; and, processing the neural signal to generate the temperature of the skin at the point of the thresholding device.

### RELATED APPLICATIONS

This application is related to and claims priority to U.S. provisional Application Ser. No. 62/110,552, filed Feb. 1, 2015, the entire contents of which are herein incorporated by reference.

### FIELD OF THE INVENTION

This application relates to the field of temperature measurement of a living body, and may be implemented before, during or after treatment of the living body for cosmetic or other treatments.

### BACKGROUND OF THE PRESENT INVENTION

### Action Potential and Neural Signal

Sensory neurons are nerve cells that transmit sensory information (sight, sound, feeling, etc.). They are activated by sensory input, and send projections (using action potential) to other elements of the nervous system, ultimately conveying sensory information to the brain or spinal cord.

Action potentials are generated by special types of voltage-gated ion channels embedded in a cell's plasma membrane. As can be seen in FIG. 1, these channels are shut when the membrane potential is near the resting potential of the cell, but they rapidly begin to open if the membrane potential increases to a precisely defined threshold value, illustrated as threshold of excitation 10 in FIG. 1. (FIG. 1 is a drawing excerpted from http://www.brynmawr.edu/math/people/vandiver/documents/HodgkinHuxley.pdf.) When the channels open (in response to depolarization in transmembrane voltage[b]), they allow an inward flow of sodium ions, which changes the electrochemical gradient, which in turn produces a further rise in the membrane potential. This then causes more channels to open, producing a greater electric current across the cell membrane, and so on. The process proceeds explosively until all of the available ion channels are open, resulting in a large upswing in the membrane potential. The rapid influx of sodium ions causes the polarity of the plasma membrane to reverse, and the ion channels then rapidly inactivate. As the sodium channels close, sodium ions can no longer enter the neuron, and then they are actively transported back out of the plasma membrane. Potassium channels are then activated, and there is an outward current of potassium ions, returning the electrochemical gradient to the resting state. After an action potential has occurred, there is a transient negative shift, called the after hyperpolarization or refractory period, due to additional potassium currents. This is the mechanism that prevents an action potential from traveling back the way it just came.

FIG. 2, a drawing figure excerpted from http://www.cns.nvu.edu/˜david/courses/perception/lecturenotes/brain/brain.html illustrates a somatosensory response, a complex of different receptors, to different strengths of stimuli—weak, moderate and strong. As can be seen, the nerve signal was acquired and recorded on the same spot 20 proximal the stimuli location 22. The nerve signal acquired shows that the 3 stimuli initiate the same amplitude of action potential (amplitude) however, there is a positive correlation between an increased stimulus strength and the frequency of such action potential generated by the stimulated group of nerve, as can be seen in the graphs 24, 26 and 28 in FIG. 2. The higher the stimulus strength, the higher the frequency of the action potential. It is at least one aspect of the present invention to use such a relation between a nerve stimulus and measured nerve response in order to estimate or evaluate a characteristic of the stimulus which initiated such response.

One can quantify a neuron's response in terms of its firing rate, the number of action potentials that occur per unit of time. For example, as can be seen in FIG. 3, a drawing figure excerpted from http://www.cns.nvu.edu/˜david/courses/perception/lecturenotes/brain/brain.html the response of a retinal ganglion cell depends on the contrast of the test light. For a dim test light we would get only a few action potentials. For a bright test light, we would get many more action potentials. Any given action potential looks exactly like all the others. When we increase the light intensity, the individual action potentials do not get bigger. Rather, we just get more of them at any given time interval.

### Modeling Action Potential

The equilibrium voltage across the neuron axon membrane for the k^{th} ion is, by convention, the intracellular minus the extracellular potential (Vₖ=Φᵢ−Φₒ). It is described by Nernst equation derived by Walther Hermann Nernst in 1888:

$${V}_{k} = - \frac{RT}{{z}_{k} F} ln \frac{{c}_{i , k}}{{c}_{o , k}}$$

$$where$$

$${V}_{k} = \begin{matrix}equilibrium voltage for the {k}^{th} ion across the membrane \\ {Φ}_{i} - {Φ}_{o} i . e . , the Nernst voltage [ V ]\end{matrix}$$

$$R = gas constant [ 8.314 J \text{/} ( mol \cdot K ) ]$$

$$T = absolute temperature [ K ]$$

$${z}_{k} = valence of the {k}^{th} ion$$

$$F = {Faraday}^{′} s constant [ 9.649 \times 104 C \text{/} mol ]$$

$${c}_{i , k} = intracellular concentration of the kth ion$$

$${c}_{o , k} = extracellular concentration of the kth ion$$

Since there are more than one ion involved, the transmembrane voltage Vₘ can be calculated using the Goldman-Hodgkin-Katz equation:

$${V}_{m} = - \frac{RT}{F} ln \frac{{P}_{K} {c}_{i , K} + {P}_{Na} {c}_{i , Na} + {P}_{C 1} {c}_{o , C 1}}{{P}_{K} {c}_{o , K} + {P}_{Na} {c}_{o , Na} + {P}_{C 1} {c}_{i , C 1}}$$

The feedback-loop of voltage-gated ion channels mentioned above made it difficult to determine their exact behavior. In 1952 Alan Lloyd Hodgkin and Andrew Huxley explained the shape of the action potential by analyzing the electrical circuit of a single axonal compartment of a neuron, consisting of the following components: 1) membrane capacitance, 2) Na channel, 3) K channel, 4) leakage current:

$${I}_{m} = {C}_{m} \frac{d {V}_{m}}{d t} + ( {V}_{m} - {V}_{Na} ) {G}_{Na} + ( {V}_{m} - {V}_{k} ) {G}_{k} + ( {V}_{m} - {V}_{L} ) {G}_{L}$$

Iₘ=membrane current per unit area [mA/cm²]
   here
Cₘ=membrane capacitance per unit area [F/cm²]
Vₘ=membrane voltage [mV]
V_{Na},=Nernst voltage for sodium, potassium and leakage ions V_{K}, V_{L} [mV]
   =sodium, potassium, and leakage conductance per unit area [S/CM²]
G_{Na},
G_{K}, G_{L}

FIG. 4, a drawing figure excerpted from http://www.bern.fi/book/04/04.htm illustrates graphically the operation of the above equation.

### BRIEF DESCRIPTION OF THE DRAWINGS

FIG. 1 illustrates the structure and operation of the action potentials of voltage-gated ion channels embedded in cellular plasma membrane when subjected to a given threshold of excitation.

FIG. 2 illustrates the somatosensory response to different levels of stimuli presented.

FIG. 3 illustrates a neuron's response when stimulated with changes in light intensity of a retinal ganglion cell.

FIG. 4 illustrates graphically the operation of the equation described in the present specification.

FIGS. 5A though 5F graphically illustrate the temperature effect on action potential.

FIGS. 6A and 6B and FIG. 7 illustrate that temperature changes of a target tissue result in changes in the action potential firing rates evoked by thermo-receptors with a living tissue.

FIG. 8 illustrates schematically one embodiment of an apparatus in accordance with the present invention.

FIG. 9 illustrates a flowchart of an embodiment of a method of operation in accordance with the present invention.

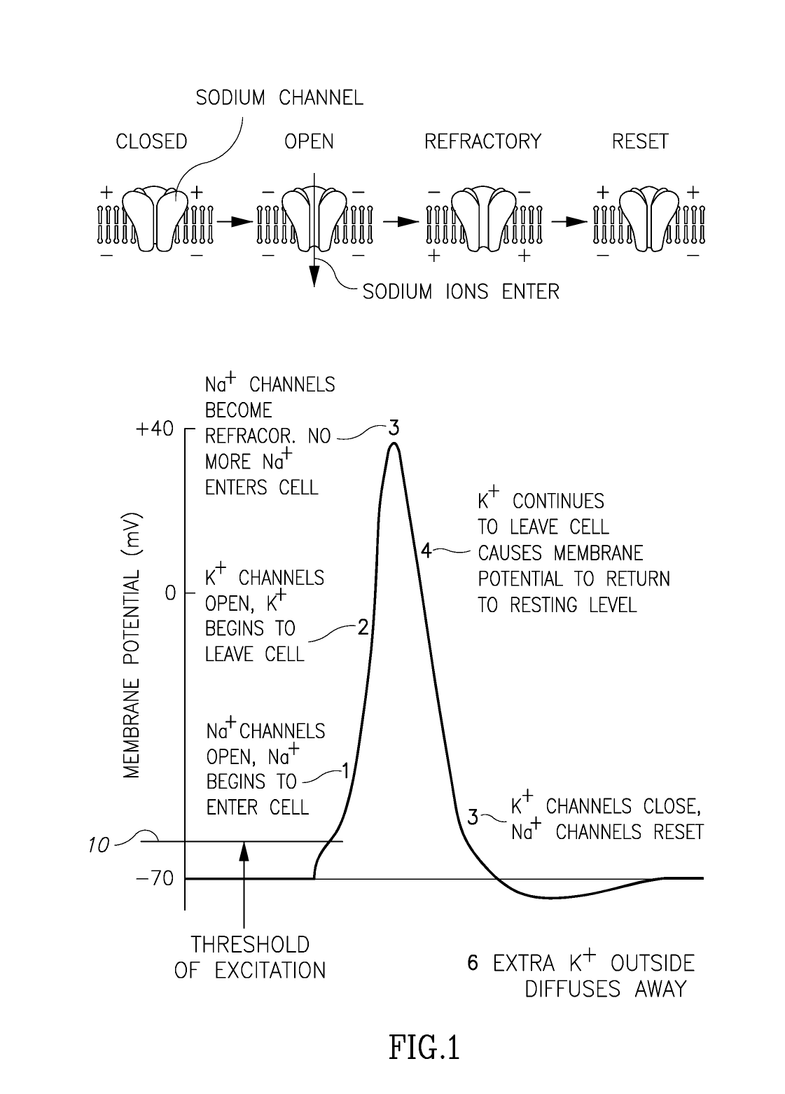

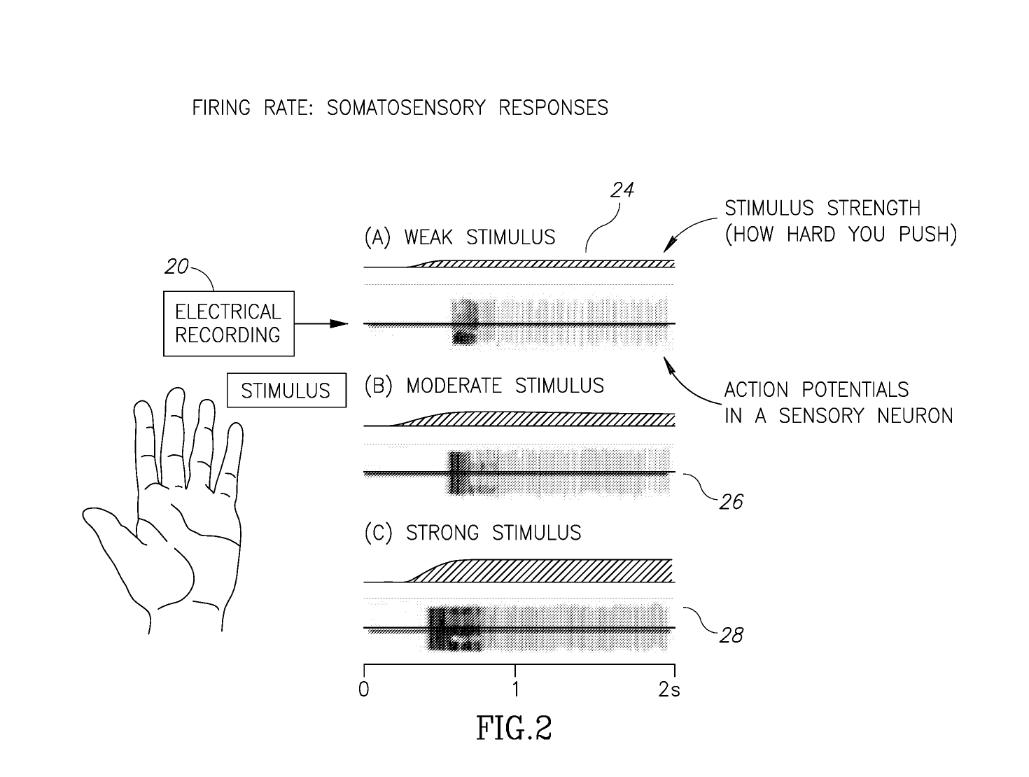

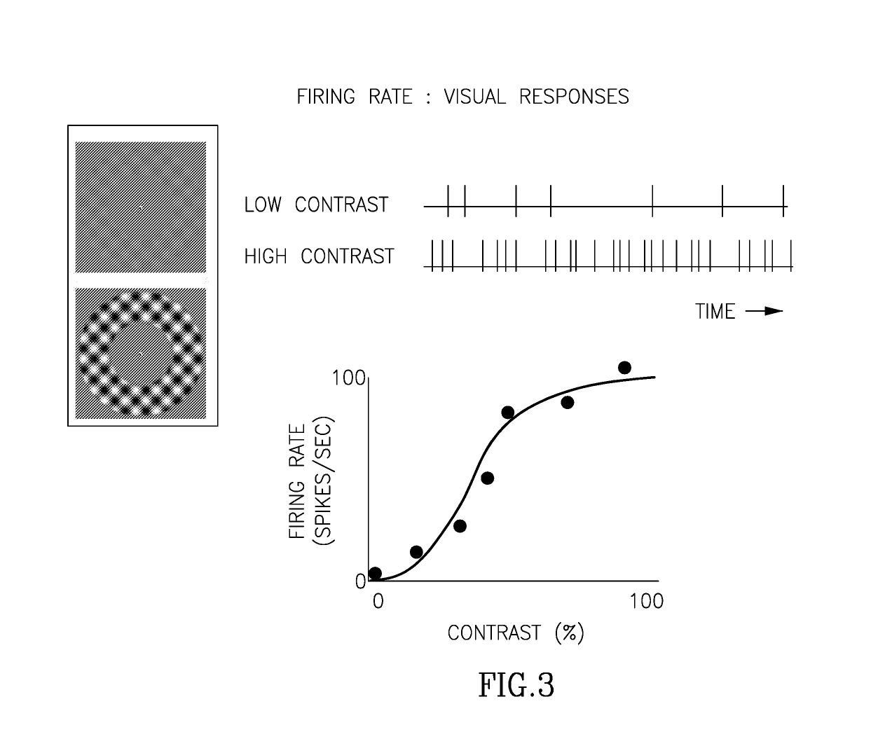

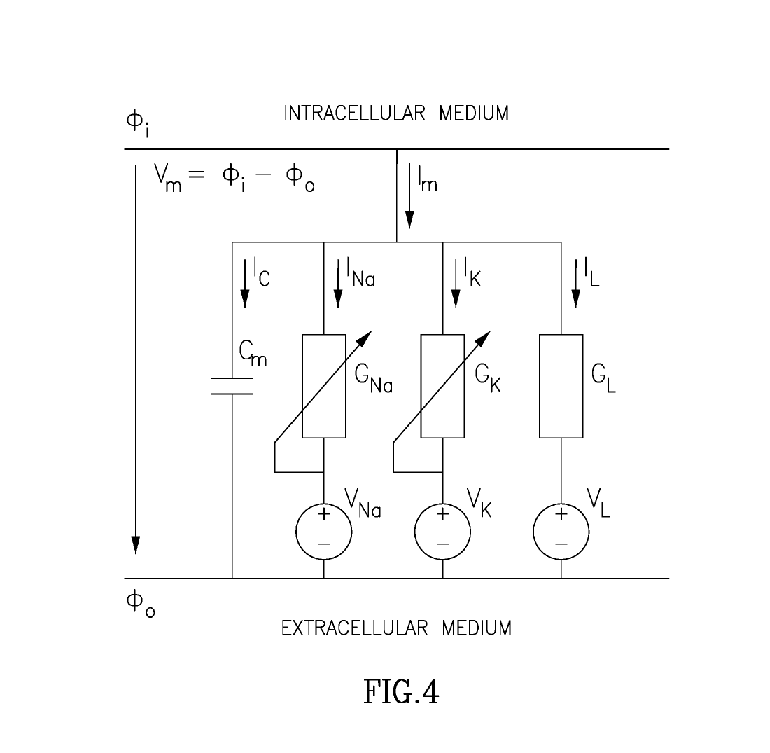

### SUMMARY OF THE INVENTION

In an aspect, a method for dynamically measuring temperature variations in skin tissue includes providing one or more electrical signal pickup elements on the skin tissue; providing at least one thresholding device to generate one or more agitating signals; providing at least one agitating device on the skin tissue at least some distance from the one or more electrical pickup signal elements; the method comprising: stimulating the skin tissue by applying one or more agitating signals to the agitating element until a neural response is indicated on a display, the neurons responding with a specific frequency modulation; identifying the relevant neural signal by locking on the modulation period; and, processing the neural signal to generate the temperature of the skin at the point of the thresholding device.

In another aspect, the method further includes the steps of: providing treatment to the skin at the point of the thresholding device, in which the treatment is of a type that causes a change in the skin tissue temperature; monitoring any variations in the neural signal frequency during the treatment; and, processing variations to determine any change in temperature of the skin tissue. The method may also include displaying changes in the skin tissue temperature.

In yet another aspect, the one or more agitating signals may be one or more of: an electrical voltage, an electric current; a mechanically driven device, a chemical device or a temperature generating device. The step of providing treatment may be a treatment head that generates EM energy to the skin tissue and the treatment head and the thresholding device may be combined in a single unit.

In yet a further aspect, a programmed controller may be provided and an indication of the temperature may cause the programmed controller to modify the operation of the treatment head.

### DETAILED DESCRIPTION OF THE PRESENT INVENTION

### Temperature Effect on Action Potential

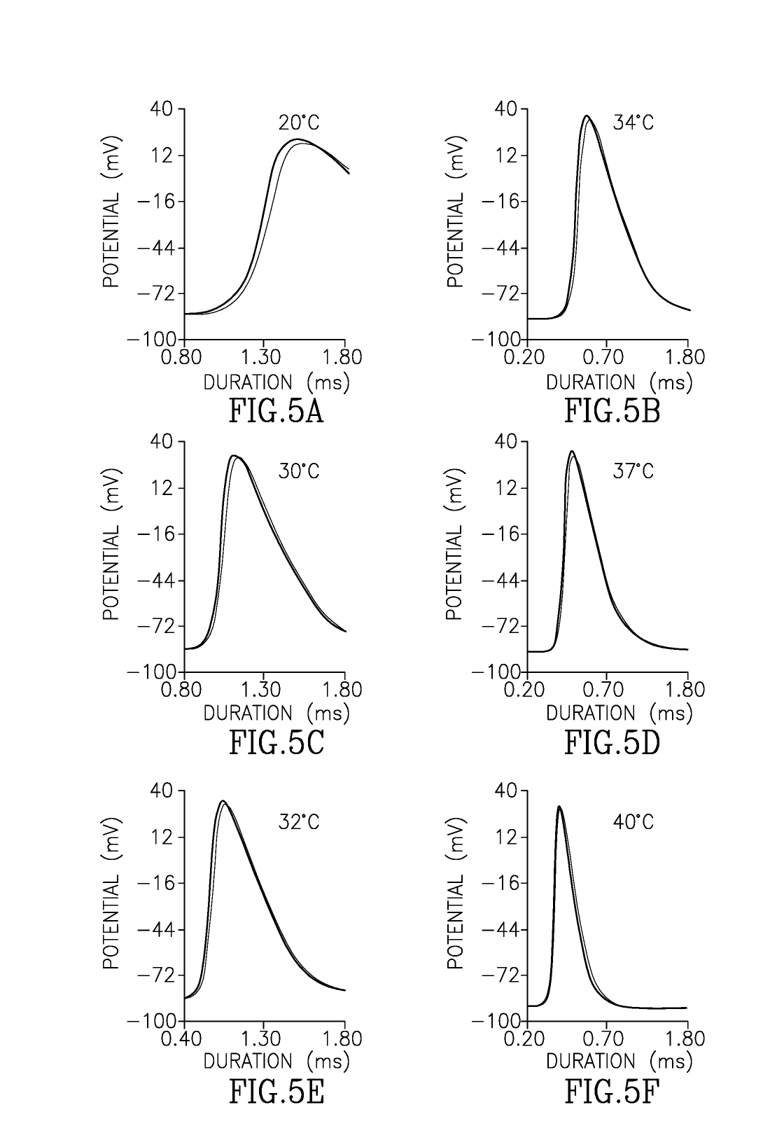

As the models of the action potential suggest, temperature change will modify the cross membrane potentials and alter the ions transfer rates across the membrane. As can be seen in FIGS. 5A through 5F, drawing figures excerpted from Stefan Krustev, Mariya Daskalova, Diana Stephanova, “TEMPERATURE EFFECTS ON SIMULATED HUMAN INTERNODAL ACTION POTENTIALS AND THEIR DEFINING CURRENT KINETICS”, Scripta Scientifica Media; Vol 45, No 4 (2013), a temperature increase will cause the slopes of the action potential to become steeper and the action potential firing rate to increase. It is one aspect of the present invention to measure and monitor changes in the slope and shape of the action potential pulses in order to monitor temperature changes of a target tissue during temperature-affecting treatments.

### Temperature Monitoring System

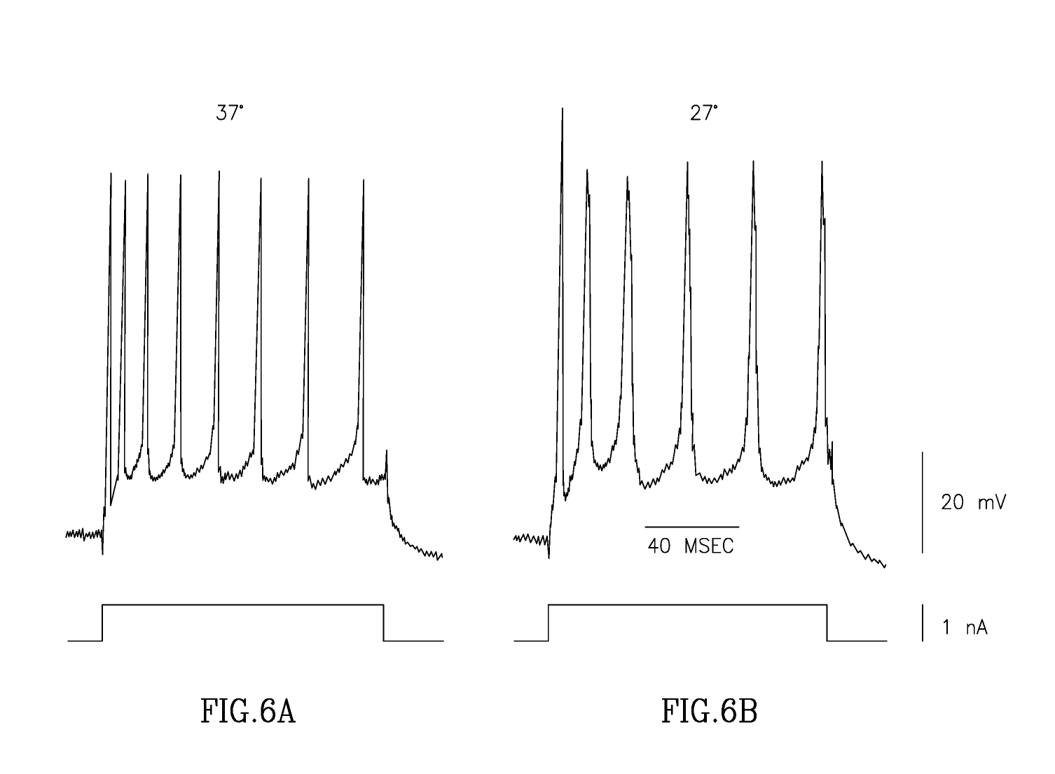

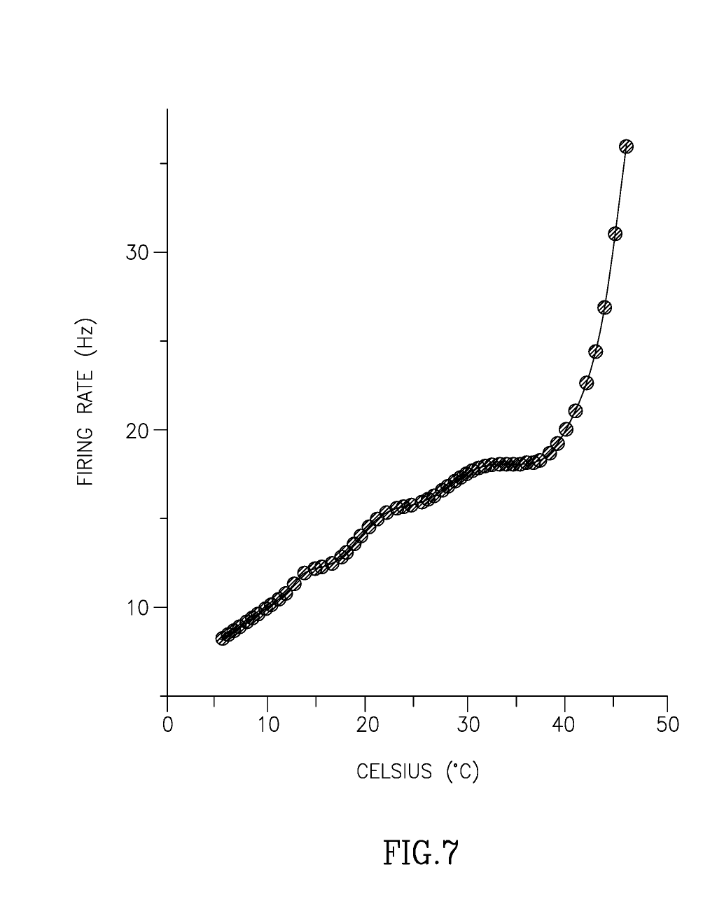

One aspect of the present invention is to monitor the temperature of a target tissue. Temperature changes of a target tissue result in changes in action potential firing rates evoked by thermo-receptors within the tissue as can be seen in FIG. 6 and FIG. 7. FIGS. 6A and 6B are figures excerpted from Scott M. Thompson, Leona M. Maskuwa, and David A. Prince, “Temperature Dependence of Intrinsic Membrane Properties and Synaptic Potentials in Hippocampal CAI Neurons In Vitro”, The Journal of Neuroscience, Vol. 5, No. 3, pp. 817-824, 1985 and FIG. 7 is a figure excerpted from Yuguo Yu, “Constant Warm Body Temperature Ensures High Response Reliability of Neurons in Endothermic Brains”, Austin J Comput. Biol. Bioinform.—Volume 1 Issue 1—2014. Temperature changes in a target tissue may result for example due to energy based treatment of the tissue. As known to those skilled in the art, energy based treatment may be for example, light or laser treatment, radio frequency, ultrasound or cold based therapy. Such treatments may target different internal or external organs of the body. According to one aspect of the invention, frequency changes of action potentials of a target tissue will be monitored in order to evaluate temperature changes of the target tissue as a result of an energy based treatment. Such energy based treatment may cause changes in the temperature of a target tissue. According to another aspect of the invention, information related to changes in the target tissue temperature will be collected, analyzed and used to feedback and control a treatment system. According to one embodiment of the invention, a proposed solution may comprise the following components:

1. A thresholding element 302 that provides a stimulus load that generates a low level stimulating signal to evoke action potential.

2. A sensing element (such as an electrode 304 and/or electrode 312) to sense the resulting neural signal.

3. A signal processing element 310 (HW and/or SW based and including a display, a memory and a programmed controller) to analyze the obtained neural signal and deduce the temperature in the stimulus environment.

4. A treatment device 306 which is utilized to treat an area 306 on the body.

The thresholding element 302 can use various stimulation type such as electric voltage, electric current, electric field, mechanical, temperature (hot or cold), chemical and any other stimulation that the neurons might be sensitive to. The stimulating mechanism may be a dedicated stimulus mechanism which is configured for this purpose only, or a combined mechanism which may provide nerve stimulation and/or treatment simultaneously or separately. Thresholding measurements, using one or more thresholding elements, may be done on a target tissue resting at a base temperature state. A base temperature state for the purpose of thresholding may be the natural temperature of the target tissue at room temperature or at any higher or lower induced temperature or set of different temperatures. Thresholding measurements of the target tissue may provide a finger-print profile for the nerve response of the target tissue at a given temperature or at selected different temperatures. During this thresholding or calibration process, the characteristic of the action potential pulses will be stored and processed within the signal processing unit 310. The slope, width and shape of the action potential as well as the correlating frequencies of the action potential firing rates at different stimuli levels and/or temperatures will be analyzed to correlate any changes in at least one of these parameters with the target tissue's temperature.

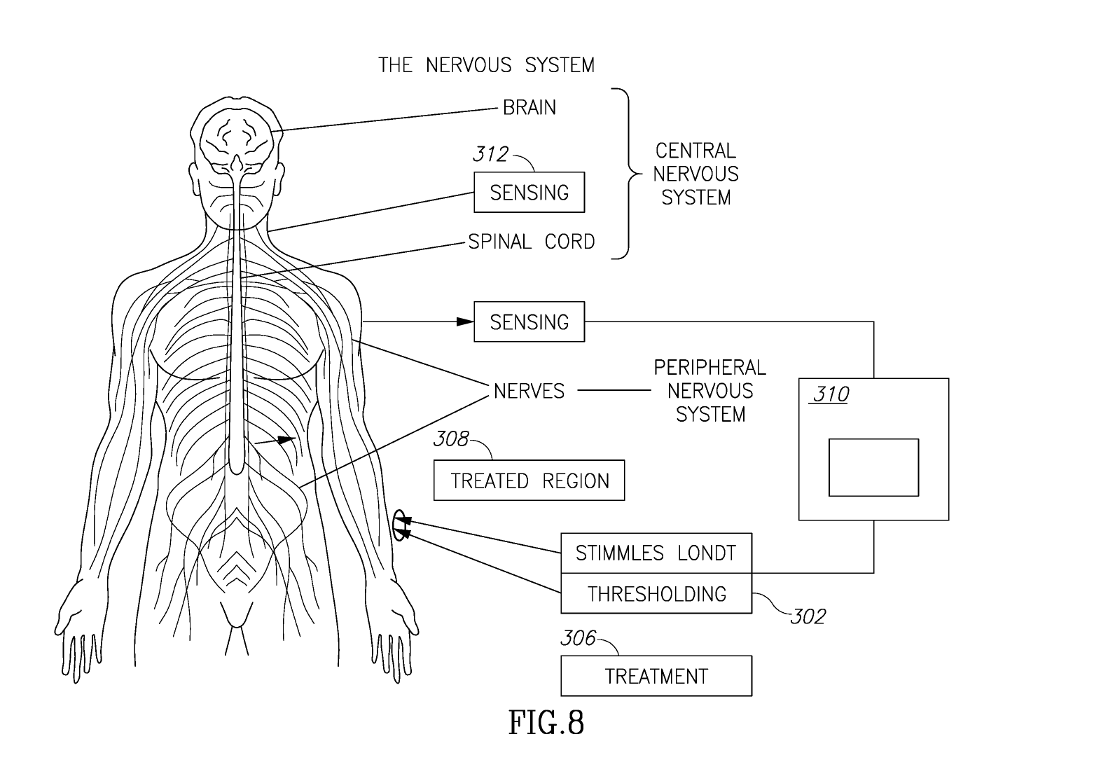

Once a neuron is stimulated, or a group of neurons, it/they will fire a series of action potentials. The firing will last as long as the stimulus is present. The firing rate depends on the stimulus level. A sensing element 304 for acquiring the nerve signal may be placed at different locations (304. 312) on the body which are proximal to the stimulus location. One example for such a location is seen in FIG. 8 as position 304. According to the aspect of the invention for measuring or monitoring changes of a target tissue's temperature, a stimulus location may be on the target tissue. Therefore, the sensing element 304 according to this embodiment may be placed proximal the target tissue 308. The sensing element 304 may be placed close to the target tissue at 308 or along nerve lines or centers more central in the body such as along the spinal cord at 312. According to another aspect of the present invention, multiple sensing elements on one location or at different locations on the body may be used in order to detect and monitor the nerve signals. It is known to those skilled in the art that recording action potentials may be difficult due to the noisy nature of the nerve signals. Therefore, according to another aspect of the invention, the stimulation intensity can vary in order to modulate the frequency (firing rate) of the action potential, and thus assist in more accurate signal monitoring and analysis.

It is also possible to introduce several stimuli that can have different modulation levels to monitor temperature variations in different parts of the body or different depth levels (such as epidermis and dermis). A modulated stimuli, according to one embodiment of the present invention, may be a thresholding stimuli. However, according to another embodiment of the invention, it can be a modulation of a treatment energy.

Once a neuron is stimulated and it fires action potential at a given rate, any variation temperature to the stimulation zone will result in a change of the firing rate. By monitoring this firing rate and analyzing its variations, one can deduce the local temperature change.

In addition to temperature, this method can be used to monitor other types of stimuli that alter the firing rate. It can be used for example to monitor local drugs and other chemicals concentration, mechanical effects, healing progress or other changes in hemostatic state of an organ.

Specifically, temperature induced treatments such as hair removal can be monitored under the present invention. For example, the temperature of the epidermis and the dermis may be monitored during laser treatment. With this information, optimized treatment parameters may be generated to increase treatment efficacy while maintaining treatment safety.

The sensing element or elements 304 and/or 312 (by way of example only) may be placed to optimize neuron signal pickup. In the event treatment on a limb is to be performed, placing an electrode at the end of the limb may be considered and implemented. Another location to place the electrode may be on the upper back or neck, above the spinal cord since all the signals from the sensory neural system go through those locations.

Once the neural signal is measured, the signal processing element 310 may be used to extract the variations in repetition rate and analyze the signal to deduce the temperature in the monitored area and to display the results on a display on unit 310.

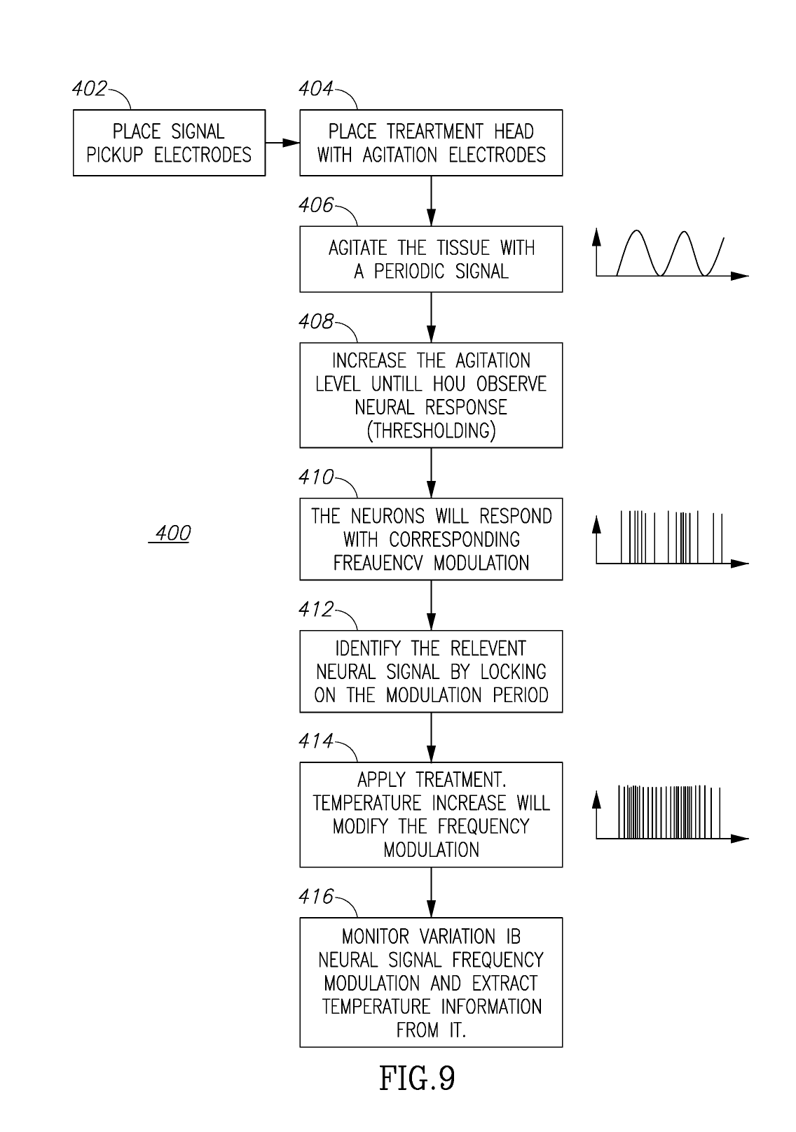

FIG. 9 is a flowchart of the process in the present invention to monitor the temperature of a target body tissue, using the various elements discussed in connection with FIG. 8.

In a first step 402, an operator or physician may place sensing/signal pickup electrodes (such as shown in FIG. 8 as 304 and 312.

In a next step 404, a treatment head, such as shown in FIG. 8 as treatment device 306 may be placed on the patient's skin surface. In this instance the treatment head 306 may additional include the stimulation load/thresholding device 302 or hey me be constituted as separate devices.

In a next step 406, the stimulus/thresholding device 302 is activated and may induce a periodic signal into the tissue.

In a next step 408, the stimulus/agitation level may be increased until the operator observes a neural response that indicates reaching a threshold level. This may be observed on a screen on device 310 or an aural or visual signal provided.

As a result of the reaching of the threshold level, the neurons will respond with a corresponding frequency modulation (410).

In a next step 412, the relevant neural signal may be identified by locking onto the modulation period.

In a next step 414, treatment may be applied with the treatment head or device 306 onto the treatment region 308. With an increase in the body temperature during the treatment process, the frequency modulation will be seen to be modified or changes. This change may be provided to the operator in any form, including a visual or aural indication provided on the display unto in unit 310.

In a next step 416, the variations of the neural signal frequency modulation from the threshold level as the temperature changes may be extracted and processed by the unit 310 to calculate and display temperature information that may be presented on the display in unit 310.

What we claim is:

1. A method for dynamically measuring temperature variations in skin tissue, the method comprising:
providing one or more electrical signal pickup elements on the skin tissue;
providing at least one thresholding device to generate one or more agitating signals;
providing at least one agitating device on the skin tissue at least some distance from the one or more electrical pickup signal elements;
the method comprising:
stimulating the skin tissue by applying one or more agitating signals to the agitating element until a neural response is indicated on a display, the neurons responding with a specific frequency modulation;
identifying the relevant neural signal by locking on the modulation period; and,
processing the neural signal to generate the temperature of the skin at the point of the thresholding device.

2. The method of claim 1, further comprising the steps of:
providing treatment to the skin at the point of the thresholding device, wherein the treatment is of a type that causes a change in the skin tissue temperature;
monitoring any variations in the neural signal frequency during the treatment; and,
processing variations to determine any change in temperature of the skin tissue.

3. The method of claim 2, further comprising displaying changes in the skin tissue temperature.

4. The method of claim 1, wherein the one or more agitating signals are one or more of: an electrical voltage, an electric current; a mechanically driven device, a chemical device or a temperature generating device.

5. The method of claim 2, wherein the step of providing treatment is a treatment head that generates EM energy to the skin tissue.

6. The method of claim 5, wherein the treatment head and the thresholding device are combined in a single unit.

7. The method of claim 5, further comprising a programmed controller and wherein an indication of the temperature causes the programmed controller to modify the operation of the treatment head.
